(19) United States
(12) Patent Application Publication
(10) Pub. No.: US 2009/0030360 A1
(43) Pub. Date: Jan. 29, 2009

(54) Film Structure With Improved Application Assistance

(76) Inventor: Axel Eckstein, Heidenheim (DE)

Correspondence Address: DREISS, FUHLENDORF, STEIMLE & BECKER, POSTFACH 10 37 62, D-70032 STUTTGART, DE

(21) Appl. No.: 12/223,531

(22) PCT Filed: Feb. 5, 2007
(86) PCT No.: PCT/EP2007/000960

§ 371 Date: Aug. 1, 2008

(30) Foreign Application Priority Data
Feb. 7, 2006 (DE) 10 2006 005 348.6

(51) Int. Cl. A61F 13/02

(52) U.S. Cl. 602/52; 602/57

(57) ABSTRACT

The invention relates to a film structure (10, 30, 40, 60) comprising a polymer film (1) and an application system enabling the film structure to be handled in a simple manner. The application system is arranged on a first side of the polymer film (1) and comprises at least one supporting film (14, 20, 34, 44c, 64c) to which at least one gripping strip (15, 25, 35, 45, 65) is applied. The supporting film (14, 20, 34, 44c, 64a) comprises a first side facing towards the polymer film (1) and a second side facing away from the polymer film (1), the second side of the support film (14, 20, 34, 44c, 64a) comprising at least one adhesive region (18, 28, 38, 48, 68).

The present invention concerns a film structure comprising a polymer film and an application system that facilitates handling of the film structure.

Application aids for plasters or wound supports have been known for some time. These application aids are used, in particular, for sheet or film structures. Film structures are thin, mostly transparent, semi-permeable sheets or films of polymer materials. The semi-permeability of the films prevents bacteria or moisture from entering, at the same time providing sufficient oxygen and water vapour exchange between the skin to be covered and the outer periphery of the film structure. These sheet or film structures are used for various purposes, e.g. as incision film for germ-free coverage of operation locations, as separate water-tight cover of wound supports that absorb exudate, and for fixing cannulas or catheters. Due to the small thickness of these sheets or films and the associated instability, these film structures are provided with the most different of application aids. Most of these application aids utilize an additional support layer which is removed during or after application of the film structure.

Sheet wound supports have also been known for some time from the patent literature. EP 81 990 B1 describes e.g. an adhesive wound support that consists of a thin polymer film. This polymer film is coated on a first side with an adhesive that adheres to the skin and, in turn, is covered by a release layer. On its other side facing away from the body during use, the polymer film additionally comprises a support layer of a fiber material that can be easily removed, e.g. an opaque non-woven material, to facilitate handling. This support layer has the same size as the polymer film.

EP 690 706 B1 describes an adhesive wound bandage having a carrier layer in order to facilitate application of a polymer film contained in the wound bandage. This carrier layer covers the overall surface of the polymer film and can be removed from the polymer film in two steps. Towards this end, in a first step, a central part is removed from the carrier layer, and subsequently, in a second step, a frame part is removed. The carrier layer of this wound bandage is disadvantageous in that it is very difficult for the user to grasp.

EP 951 263 B1 describes an adhesive film structure, the adhesive side of which is covered by a protective layer that consists at least of two parts and can be removed, and the non-adhesive second side of which has a one-piece support layer over its entire surface. In this film structure, the support layer is connected on two opposite sides to the protective layer in a hinged fashion, such that the support layer can be simultaneously removed with removal of the protective layer.

EP 985 391 A1 describes a bandage material on a film basis, which has a non-adhesive grasping area in the edge area of the film. The non-adhesive side of the film has a one-piece support layer that has the same size as the film and comprises at least one gripping strip. By pulling the grasping area in the direction of adhesion, the applied film can be removed without causing pain.

European patent document EP 630 628 B1 discloses a sheet structure that comprises a two-part support film in order to facilitate application. This support film is larger than the sheet to be applied and covers it completely. In order to remove the support film, the support film comprises a further adhesive removing strip which is disposed above the line of intersection of the support film and has two non-adhesive edge areas to be gripped, which serve as a gripping strip. Only part of the support film can be removed by means of this further removing strip, with the result that the second part of the support film remains on the polymer sheet.

European patent EP 341 045 B1 describes an adhesive transparent or translucent film structure that comprises a transparent wound support and a transparent film that is provided with reference numerals. These reference numerals may be used for monitoring the wound.

All of these property rights show different alternative solutions for sheet or film structures with different application systems. The film structures proposed by these property rights can be regarded as having too complicated a structure or being difficult to handle.

It is the object of the present invention to provide a film structure that enables secure handling and also comprises means for documenting application thereof. An additional object is to provide a film structure which can be precisely mounted onto the intended surface, can fix a therapeutic means, such as e.g. a cannula, and enables and ensures simple documentation of use of the film structure or of the therapeutic measure itself, during routine operation. The film structure should thereby be universally usable without any limitation with regard to shape or size.

This object is achieved by a film structure comprising a polymer film and an application system for facilitating handling of the film structure, wherein the application system is disposed on a first side of the polymer film and comprises at least one support film to which at least one gripping strip is additionally formed, wherein the support film has a first side facing the polymer film and a second side facing away from the polymer film, the second side of the support film comprising at least one adhesive area.

In a second preferred embodiment, the support film comprises an adhesive area that is covered by a covering means, in particular, a strip-shaped covering means.

One advantage of a film structure comprising an application system of this type consists in that the support film can be simultaneously used as a documentation means. This documentation is helpful for handling film structures, since the film structures are usually applied for a time period of 7 to 10 days. During use of an inventive film structure, the support film that is removed during application can be glued into a patient or documentation file either directly or after removal of the covering strip through and by means of the adhesive area. This provides a film structure in a particularly simple fashion, which can be applied in a particularly simple fashion by the gripping strip, and at the same time provides the user with a means for documenting the application of the film structure. Moreover, unambiguous documentation is possible in order to prevent premature or belated change of the film structure.

The application system of the inventive design includes any means that facilitate application of the polymer film and comprise at least one support film and, in addition to the support film, at least one gripping strip that is formed on the support film. “Formed on” thereby means two identical or two different materials which are connected to each other in a detachable or undetachable fashion by means of adhesive, pressure, thermal energy, ultrasound methods or other methods. In the present case, the gripping strip is always an additional material component, wherein the gripping strip can always be simultaneously removed from a polymer film together with at least one support film. For reasons of clarity, a sheet or polymer film in accordance with the present invention refers to the sheet or polymer film that is to be actually applied, such as e.g. a wound bandage. In contrast thereto, a sheet or polymer film or support film is defined as part of the application system, i.e. the sheet and film are merely distinguished in view of the function of the components. With respect to the material, the terms “sheet” and “film” can be used interchangeably, according to normal linguistic usage. Documentation means the collection and temporary archiving of information.

The expert is quite free with respect to the design of the adhesive area on the support film. This adhesive area may e.g. be realized by simply applying a pressure-sensitive adhesive in the form of strips or points. Adhesives that allow removal or repositioning of the support film in a patient file are particularly suitable. A double-sided adhesive tape may also be used, wherein the adhesive tape is non-detachably connected to the support film using the first adhesive surface, and the adhesive second side opposite to the first side forms the adhesive area of the support film. This embodiment has turned out to be particularly feasible when the second side of the double-sided adhesive tape has particularly good adhesive properties on paper. The use of adhesives having an adhesive force of 0.05 to 0.5 N/5 mm (measured according to DIN EN 1939) is particularly favorable.

In one preferred embodiment of the invention, the gripping strip may be disposed on the support film using the adhesive area. In this embodiment, the gripping strip forms the covering strip. It must thereby be ensured that the gripping strip can be removed from the support film without deforming or destroying the support film. Moreover, the adhesive force between the gripping strip and the support film is sufficient in order to completely remove the support film from the polymer film using the gripping strip. An adhesive that is disposed on and forms the adhesive area must e.g. also completely remain on the support film after removal of the gripping strip in order to be able to glue the support film directly into a patient file without further modification. If the cover layer is made from a different material than the gripping strip, these requirements must also be met. Any conventional silicone film or paper and a film or paper loaded or loadable with a fluorine compound may thereby be used as the covering strip.

For universal application of the support film, the support film itself can be inscribed on its first side facing the polymer film using a conventional writing means, e.g. a permanent marker. The user can provide documentation on the support film concerning the person to be treated or the time of application. In particular, the first side should be writeable, since this side will be lying on top after it has been glued e.g. into a patient file. In this fashion, writing thereon is still possible after it has been glued into a patient file.

For reasons of clarity, the support film may be provided with an imprint that provides information for facilitating documentation of use. This imprint may show e.g. the words “date”, “user”, “patient” or the name of the producer or the product name. The imprint may be disposed on the first side of the support film facing the polymer film or on the second side of the support film. If it is printed on the second side, the imprint will be made in a mirror-inverted fashion to ensure that it can be read after gluing it into a patient file.

Moreover, a multi-layer film or a film structure may be used as a support film. These multi-layer films are advantageous in that a second film layer may cover any possible imprint on a first layer of the film. It is then possible to provide a marking with a permanent marker on an outer surface of the film that has a regular and uniform shape. The overall film may be colored, in particular, in such a fashion that it is nevertheless transparent or translucent. Transparent or translucent support films are particularly preferred.

The invention also concerns the use of a support film of a film structure for documentation of application of the film structure.

In a further particularly preferred embodiment, the film structure comprises a polymer film that comprises at least one first area without support film. Due to the first area without support film, the surface of the polymer film covered by the support film is smaller than the surface of the first side of the polymer film. The film structure comprises, in particular, one or more support films whose overall contact surface is less than 97% and, in particular, less than 94% of the surface of the first side of the polymer film to be applied.

One advantage of a film structure of this type with application system consists in that the area of the polymer film without support film, i.e. the area that is not covered by a support film, can work as a joint during application of the film structure due to the improved flexibility compared to the polymer film with support film. For this reason, a relatively rigid support material may also be used as the support film, which ensures at the same time a perfect fit. However, with respect to a conventional one-piece support film, which has the same size as the polymer film or with respect to a multi-part support film which, in total, has the same size as the polymer film, the selection of the support material must be limited to relatively flexible materials in order to ensure application of the polymer film with perfect fit. Another advantage is that the amount of material that is used for the support films is reduced.

In particular, a first area without support film may be disposed on an edge of the polymer film. The edge thereby means any area of a surface that extends from the edge of a surface to the inside of a surface, wherein the surface extension of the edge is less than 50% of the entire surface. This provides a film structure which advantageously comprises an area having a flexibility that is predetermined by the polymer film itself, and ensures easy initial fixing of the film to be applied, wherein at the same time, the support films on the further areas ensure safe handling. It has thereby turned out to be particularly advantageous for handling and application safety when at least one point of the outer edge of one of the support films is spaced apart from the outer edge of the polymer film by at least 2 mm, in particular at least 3 mm, and with particular preference at least 5 mm. A constant separation of at least 2 mm, in particular at least 3 mm, and with particular preference at least 5 mm, from the outer edge of the polymer film at any point of the edge of the support film is particularly preferred.

In a further embodiment of the invention, a gripping strip completely extends over an area of the polymer film that is not covered by the support film. In this case, the overall application system may have the same size as the polymer film. The same size thereby means a size with respect to the contact surface, i.e. the peripheral border of the application system and of the polymer film are flush. Since the application system and the polymer film have the same size and since the gripping strip is formed only on the support film and is not connected to the polymer film, it is ensured that, in addition to the above-described high flexibility in the area that is not covered by the support film, the entire film is additionally covered and is thereby completely protected even before and during application.

In one preferred embodiment of an inventive film structure, a gripping strip has a gripping surface which is determined to be gripped by the user, and is preferably designed as an engagement means, of at least 2 cm², in particular at least 5 cm² and with particular preference at least 7 cm².

In a further embodiment of this invention, the application system comprises at least two support films which are disposed in a plane parallel to the polymer film. In such an embodiment of the invention, a first area without support film can be disposed between the first and the second support film. The separation between these two support films at any point is thereby preferably at least 2 mm, in particular 3 mm and with particular preference at least 5 mm. An application system is particularly preferred which has two support films that have identical separation from each other at any point.

In particular, in the embodiment of the film structure comprising two support films, each support film comprises one gripping strip. A first gripping strip is thereby associated with the first support film and a second gripping strip is associated with the second support film.

In one preferred embodiment of this inventive film structure comprising two support films, the first gripping strip comprises a gripping surface determined to be gripped by the user, preferably designed as an engagement means, and the gripping surface of the first gripping strip extends over at least sections, in particular, the overall second gripping strip.

This arrangement of the gripping strips ensures, in a particularly simple fashion, that the applicant can, in any case, only actuate the uppermost first gripping strip as a first gripping strip, and is thereby able to remove as a first film, a first support film from the polymer film. Only in the second step is the applicant then able to remove a second support film using the second gripping strip. The support films are therefore removed in a certain order and a film structure is provided, which offers particularly safe handling.

The size of the gripping surface of the first gripping strip is preferably at least 2 cm², in particular at least 5 cm², and with particular preference at least 7 cm². Handling safety is particularly improved when the first gripping strip has a free gripping surface of at least 2 cm², in particular 4 cm², and with particular preference 6 cm². In the present case, this free gripping surface is that part of the gripping surface that laterally projects past the second gripping strip.

Handling is particularly safe when the first gripping strip completely extends over the area of the polymer film without support film and also over the second gripping strip.

When the film structure comprises an application system with two support films, and a first area without support film is provided between the support films, a second area without support film can be provided separately from this first area without support film. A gripping strip may moreover preferably extend over the second area, or this second area may not be covered by a support film or a gripping strip. This second area of the polymer film without support film is preferably disposed at an edge of the film structure. In this fashion, the film structure has a joint within the structure and also an area for initial fixation.

When an application system is provided which comprises more than two support films, each support film may be associated with one gripping strip. In a particularly preferred embodiment, one gripping strip may also be associated with two support films. In a film structure comprising three support films, one gripping strip may, in particular, be associated with two support films. With this arrangement or association of the gripping strips on or to the support films, two separate support films can be removed in one working step.

An inventive film structure may have any geometrical shape, such as e.g. oval, square, rectangular, pentagonal, hexagonal or other feasible shapes. An inventive film structure may, in particular, also have a rectangular shape. In accordance with a further preferred embodiment, an inventive film structure may have an incision extending from an edge area to a central area. In such an embodiment, the present film structure can be used with particular preference as a so-called cannula or catheter plaster.

In particular, transparent or translucent film materials are provided as support films. Alternatively, opaque or non-transparent film materials may also be used. In particular, films that are produced from polyester, polyethylene, polypropylene, polyvinyl chloride, polystyrene, polyamide, polycarbonate, cellulose ester, ethylene vinyl acetate, polyvinyl acetate, polyvinyl alcohol and/or mixtures thereof may be used as support film. Support films of transparent polyester or polyethylene or polypropylene are particularly preferred. It has thereby turned out to be particularly advantageous for the support film to have a thickness of 15 to 80 μm, in particular 20 to 60 μm, and with particular preference 20 to 40 μm.

A gripping strip may be produced from the same materials as the support film. In one particularly preferred embodiment, the gripping strip is produced from a film material which is more flexible than the support film. When an application system is provided which comprises two or more support films and two or more gripping strips, preferably all gripping strips are produced from a material that is more flexible than any support film. This ensures that the gripping strips are particularly easy to grasp. In a further particularly preferred embodiment with two gripping strips, the gripping strip of the first support film is more flexible than the gripping strip of the second support film. It is thereby also advantageous that the first gripping strip completely extends over the second gripping strip.

In addition, in a system with two gripping strips, an activation means may be provided that is disposed between the first and the second gripping strip. This activation means may e.g. be an additional adhesive strip which has a different adhesive force on each side of its contact surfaces. When a film structure of this type is used, a first gripping strip that is disposed above the second gripping strip may be grasped and the activation means and a support film may be removed from the polymer film using this gripping strip, wherein at the same time, the second gripping strip is activated or prepared in such a fashion that grasping is facilitated for the user.

In an inventive film structure, in particular, polymer films may be used as the polymer film, which have a high water vapor permeability. In this connection, sheets are particularly suited which are produced from polyurethane, polyetherurethane, polyester urethane, polyether polyamide copolymers, polyacrylate or polymethacrylate. A polyurethane film, polyester urethane film or polyether urethane film are particularly preferred as polymer film. Polymer films that have a thickness of 15 to 50 μm, in particular 20 to 40 μm, and with particular preference 25 to 30 μm are also particularly preferred. The water vapor permeability of the polymer film in an inventive film structure is preferably at least 750 g/m²/24 h, in particular, at least 1000 g/m²/24 h and with particular preference at least 2000 g/m²/24 h (measured according to DIN 13726).

A glue may be disposed on the second side of the polymer film to be applied opposite to the application system. It may be applied either on the entire surface as well as discontinuously or only in certain areas. The adhesive used may be a conventional adhesive, in particular, an acrylate adhesive. With particular preference, the adhesive is a hydrogel adhesive, in particular, on the basis of aqueous acrylates.

The surface density of the adhesive is preferably 20 to 100 g/m², in particular 30 to 50 g/m², wherein the adhesive may be disposed discontinuously, but preferably over the entire surface.

The water vapor permeability of the polymer film provided with the glue is preferably at least 1000 g/m²/24 h, with particular preference at least 1200 g/m²/24 h, and with particular preference at least 2000 g/m²/24 h (measured according to DIN EN 13726).

In accordance with a further development of the invention, the film structure may be coated with an adhesive on its entire surface on the second side of the polymer film opposite to the application system, and the adhesive may be protected with a cover foil or cover paper. Any conventional silicone paper or film and a paper or film loaded with a fluorine compound may thereby be used as the cover layer.

In order to provide a film structure that can be safely applied, the materials that are used must be precisely adjusted. The materials used must, in particular, be adjusted with respect to their release properties. These release properties which can be adjusted by additional means are based on the forces that act between the two materials. By selective surface treatment of a material, one can generate an attracting or repelling effect on a second material to be combined with the first material. Surface treatment that causes an attracting effect between two materials may be performed e.g. by additional adhesive application, static charging or melting the two materials to be combined. A repelling effect can be generated e.g. by an additional layer on a material of silicone or fluorine compounds. The release force (removing force) thereby means the force which is required to separate two materials from each other (measured according to DIN 53530).

In a further embodiment of the inventive film structure, these release properties are adjusted in such a fashion that the removing force required to remove a cover foil or paper from the polymer film to be applied is larger than the removing force required to separate the support film or films from the polymer film.

In one design of the film structure with two support films, the release properties are preferably adjusted in such a fashion that the removing force required to release the first support film from the polymer film to be applied is as large as the removing force required for removing the second support film.

The release properties of a film structure with two support films and two gripping strips are preferably adjusted in such a fashion that the removing force required to release the first gripping strip from the second gripping strip or the second from the first gripping strip is smaller than the release force required to release the support film from the polymer film to be applied.

In a further embodiment of the film structure with two support films, the release properties are preferably adjusted in such a fashion that the removing force required to remove the first support film from the polymer film to be applied is larger than the removing force required to remove the first gripping strip from the second gripping strip.

The adhesion of the support film on the polymer film is preferably only 0.01 to 0.5N/25 mm, with particular preference 0.01 to 0.1 N/25 mm, measured according to DIN 53530. Towards this end, the support material is preferably disposed onto the polymer film directly during production of the polymer film, or the polymer film is generated directly on the support material. All conventional methods for producing a film may be applied, e.g. by casting, doctoring, extrusion or other conventional film or sheet production methods. If required, the support material may be roughened on the coating side or be subjected to a different treatment that improves adhesion. A coating that enhances adhesion may also be advantageous. It is thereby important that the adhesion of the polymer film on the surface to be applied is substantially larger than the adhesion of a support film on the polymer film.

In a particular embodiment of the invention, a film structure comprising a polymer film with an application system is disposed in a package. The package is thereby a sterile package. A wound pad may also be separately inserted in the package in addition to the film structure.

The invention is explained below with reference to the drawing. In the drawing:

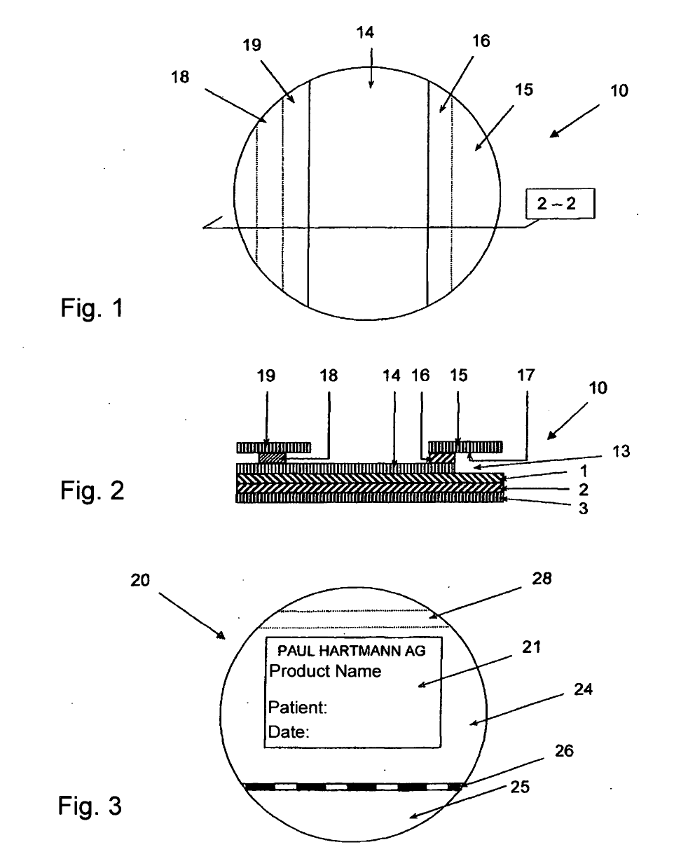

FIG. 1 shows top view of a first embodiment of a film structure;

FIG. 2 shows a cross-sectional view of the embodiment of a sheet structure represented in FIG. 1;

FIG. 3 shows an embodiment of a support film as information carrier;

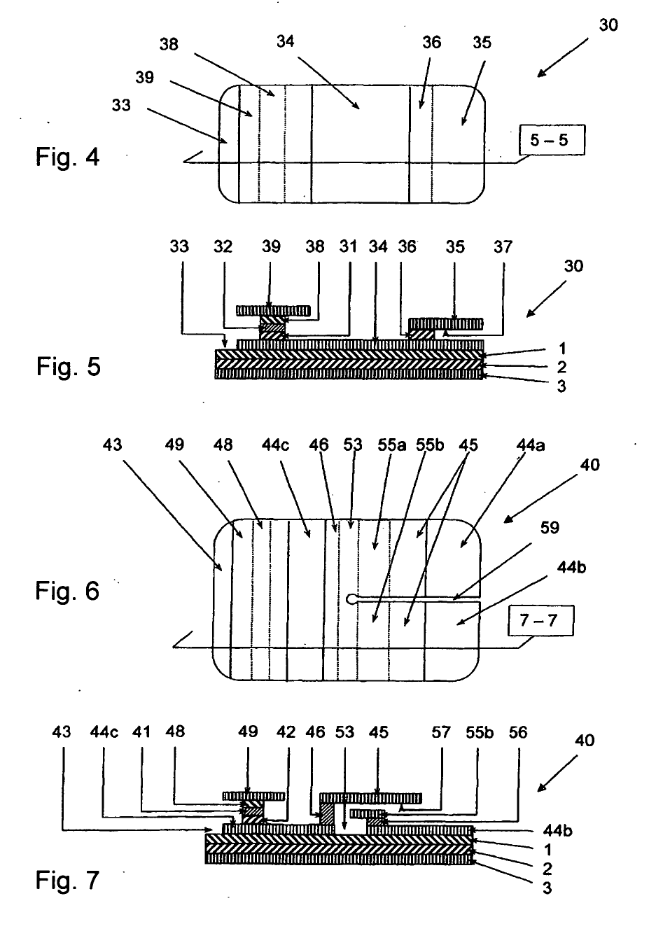

FIG. 4 shows a top view of a further embodiment of a film structure;

FIG. 5 shows a cross-sectional view of the embodiment of a film structure represented in FIG. 4;

FIG. 6 shows a top view of a further embodiment of a film structure;

FIG. 7 shows a cross-sectional view of the embodiment of a film structure represented in FIG. 6;

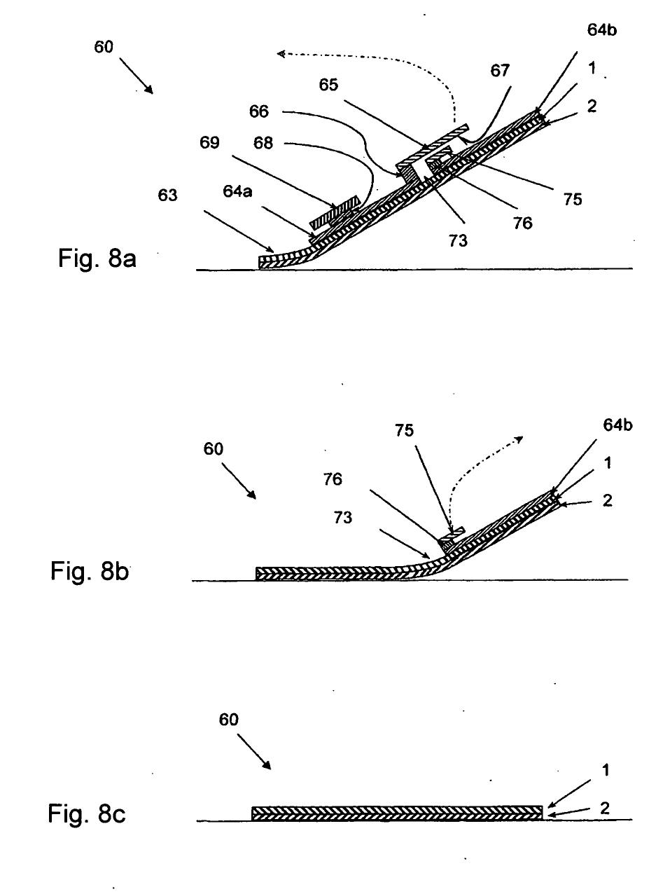

FIGS. 8a-c show cross-sectional views of an embodiment during use.

FIGS. 1 and 2 show a first embodiment of an inventive film structure. All dotted markings therein and in the following define the borders of the components which lie below the components represented with solid lines. The overall film structure (10) is circular. It consists of a transparent polymer film (1) that is covered with an application system on its first side. The second side facing away from the application system is provided with an adhesive glue (2) that is protected by a cover layer (3). The application system consists of a likewise transparent support film (14) which completely covers the polymer film (1) except for the edge area (13) which is without support film. The support film (14) moreover comprises a gripping strip (15) which is firmly connected to the support film by an adhesive (16). The gripping strip is not connected at any point to the polymer film and completely covers the edge area (13) without support film. The user can grasp the gripping strip (15) with particular ease due to the gripping surface (17). The overall application system therefore has the same size as the polymer film (1) and has the same peripheral border as the polymer film. The support film moreover comprises an adhesive area (18) on the side facing away from the polymer film, which is covered by a cover strip (19) of a siliconized paper. During use, the applicant can remove the siliconized cover strip (19) after application of the film structure (see FIGS. 8a-8c), and glue the support film into a patient file for documentation of use of the film structure.

FIG. 3 shows a top view of an example of a support film (20), wherein this representation shows the top view of the first side facing the polymer film in the film structure. This support film consists of a single-layered translucent polyethylene polymer film (24) to which a gripping strip (25) of a colored translucent polyethylene material is disposed using an ultrasound seam (26). An imprint is disposed on the first side of the support film. This imprint characterizes an information field (21) for taking over data concerning the use of the product. The polymer film can be glued onto the lower side of the film in a patient file using an adhesive area (28).

FIGS. 4 and 5 show a second embodiment of the present invention. This film structure (30) has a rectangular basic shape and consists, like the first embodiment, of a polymer film (1), a full-surface glue deposit (2) disposed on the polymer film, and a cover layer (3) covering the glue. On its first side, the polymer film comprises an application system comprising a support film (34), a gripping strip (35) disposed on the support film using an adhesive (36), and an adhesive area (38). The adhesive area (38) is realized by a strip that is adhesive on two sides and consists of a first adhesive (31), a carrier material (32) and a second adhesive (38). The first adhesive ensures firm connection between the carrier material (41) and the support film. The adhesive (38) covered by a siliconized paper (39) forms the adhesive area of the support film (34) by means of which the support film can be fixed in a patient file etc., providing documentation of the application of the film structure. The overall support film is smaller than the polymer film, i.e. the contact surface of the support film corresponds to 95% of the contact surface of the polymer film. Since the area without support film forms an edge area of the polymer film, this area can be used for application of the film structure for initial fixation.

FIGS. 6 and 7 represent a further film structure (40). This film structure can be used as a cannula or catheter plaster. The film structure has a rectangular basic shape, the short side of which has a slit (59) that is parallel to the long side. This slit provides the film structure with two independent areas that are connected to each other via a third area, and can each be fixed on a surface on one side of the cannula during fixation, e.g. of a cannula. The film structure has a polymer film (1), an acrylate adhesive layer (2), and a cover layer (3) that covers the adhesive layer. An application system is disposed on the first side of the polymer film facing away from the adhesive layer. This application system comprises three gripping strips (45, 55a, 55b) which are disposed onto three support films (44a, 44b, 44c) using three adhesives (46, 56a, 56b-56a is not shown). The first gripping strip (45) which is associated with the first support film (44c) extends over the central area (53) of the polymer film, which is not covered by support films, and also the second and third gripping strips (55a, 55b). The outer part of the gripping surface of the gripping strip (45) laterally projects as a free gripping surface (57) over the second and third gripping strips (55a, 55b), such that the order for removing same is predetermined. The gripping strip (45) is moreover incised along part of its surface by the slit (59). The second and the third gripping strips (55a, 55b) also extend over a central area of the polymer film (53), which is not covered by a support film. An adhesive tape is disposed on the first support film (45), which is adhesive on two sides, and consists of a first adhesive (42), a carrier material (41) and a second adhesive (48). The adhesive (48) covered with a siliconized paper (49) forms the adhesive area of the first support film (44c) by means of which the support film (44c) can be fixed in a patient file etc. This enables documentation of application of the film structure.

The support films (44a, 44b, 44c) cover an overall surface of the polymer film that corresponds to 92% of the surface of the first side of the polymer film, since in addition to the central area (53) that is not covered by the support films, the edge area (43) also does not have a support film. This edge area can be used for initial fixation after removing the cover layer. Moreover, an activation means may be provided between the two gripping strips. This activation means may be a double-sided adhesive tape that has a larger adhesive force for the first gripping strip (45) than for the second gripping strip (55a, 55b). During removal of the support film (44c) using the first gripping strip (45), the second and third gripping strips (55a, 55b) disposed below are thereby prepared before the adhesive force between the second and the third gripping strips (55a, 55b) and the activation means is eliminated. The second and the third gripping strips are thereby easier to grip in the next step for removing the second and third support films (44a, 44b).

An inventive film structure (10, 30, 40, 50) is applied by initially removing the cover layer (3) from the adhesive layer (2). As is shown in FIGS. 8a through 8c, in a film structure with two areas without support film, a first edge area (43, 63) without support film may be fixed for applying a film structure (40, 60). This is easy to realize due to the high flexibility of the polymer film in the area without support film. In a further step, the applicant can exactly position the polymer film (1) to be applied due to the predetermined arrangement of the gripping strips (45, 55, 65, 75) which are rigidly disposed on the respective support films using adhesive glues (46, 56, 66, 76). Due to the second area (53, 73) without support film, the film structure has a type of joint that permits application without generating folds. The support films (44a, 44b, 44c, 64a, 64b) may be removed one after the other during application or after application of the polymer film, wherein the first gripping strip (45, 65) is initially grasped via its free gripping surface (57, 67), thereby initially removing the first support film (44a, 64a).

### EMBODIMENT 1

The film structure has a rectangular basic shape with an edge length of 105×85 mm (contact surface 89.25 cm²). It comprises a transparent polyurethane film which is coated on its side facing the body during use with a pressure-sensitive acrylate adhesive. The adhesive is disposed onto the polymer film having a thickness of approximately 25 μm (measured with a test pressure of 0.5 kPa) through full-surface application in an amount of approximately 25 g/m². The polymer film and the adhesive together have a water vapor permeability of approximately 1200 g/m²/24 h (measured according to DIN EN 13726). Such a polymer film can be obtained under the trade name Inspire 1305 from the company InteliCoat Technologies, Wrexham Industrial Estate, Wrexham LL139UF,UK. The adhesive side of this polymer film is covered by a siliconized cover paper available under the trade name Separacon 980-60 from the company Maria Soell GmbH & Co. KG, Frankenstrasse 45, D-63667 Nidda-Eichelsdorf. The film structure is produced as a cannula or catheter plaster and has the structure that is basically shown in FIGS. 6 and 7. An application system is disposed on the other side of the polymer film, facing away from the body during use, which consists of three support films of polyethylene terephthalate (PET), on each of which one gripping strip is disposed. The present film structure contains a polymer film having two separate areas which are not covered by a support film or a gripping strip. This is an edge area (see FIG. 6 or 7, (43)) and a central area (see FIGS. 6 and 7, (53)) of the polymer film. The edge area is disposed on the short side of the rectangle, and has a uniform width of 6 mm. The two support films which are separated by the slit have the same size and have an edge length of 42×42 mm (contact surface: 2×17.6 cm²=35.3 cm²). The separation between the two films at any point of their edges, that have identical lengths and are disposed parallel to each other, is approximately 1 mm. This separation corresponds to the gap (59) shown in FIG. 6. The third support film has a contact surface of 85×50 mm (contact surface. 42.5 cm²) and is spaced apart from the two other support films by 10 mm via the central area (53) on the long side. This yields a total contact surface of the three support films of approximately 87% of the surface of the first side of the polymer film. The support films are produced from a PET film having a thickness of 30 μm (measured at a test pressure of 0.5 kPa). A gripping strip is glued onto each support film using an acrylate adhesive. The arrangement of the gripping strips is represented in cross-section in FIG. 7, wherein the first gripping strip shown with reference numeral (45) has a size of 85×30 mm, and is connected over the entire width (85 mm) to the associated support film. The second gripping strip (reference numeral 55a) and the third gripping strip (reference numeral 55b) each have a size of 42×15 mm. All gripping strips are connected to the respective support film via an adhesive connecting strip having a width of 4 mm, and are produced from a transparent polyester film having a thickness of 20 μm. The first gripping strip thereby has a gripping surface with a uniform width of 26 mm. The size of the gripping surface of the first gripping strip is 85×26 mm=22.1 cm². The uniform width of the gripping surface of the second and third gripping strips is 11 mm. The size of the gripping surface of the second or third gripping strip is therefore 42×11=4.6 cm². The uniform width of the part of the first gripping surface that projects past the second or third gripping strip, i.e. the width of the free gripping surface of the first gripping strip is 12 mm. The size of the free gripping surface (57) is thereby 10.2 cm². An adhesive tape of a thickness of 0.08 mm is disposed on the first support film (44c, see FIGS. 6 and 7), which has a width of 5 mm and is adhesive on both sides. This adhesive tape consists of a transparent polyester carrier film (41) that has an acrylate adhesive disposed on both sides. The first side (42) of this double-sided adhesive tape is disposed onto the support film. The second side (48) forms the adhesive area of the support film. This adhesive area is covered by a silicone paper having a surface density of 92 g/m² and a thickness of 0.077 mm. An adhesive tape of this type can be obtained from the company SwissChem Ag, Widnau, Switzerland. The name of the producer, the product name and the open marking fields “date” and “patient” are disposed on the writeable support film. The adhesive force of the adhesive area of the support film is 0.16 N/5 mm with respect to paper (80 g/m², woodfree, ultra-white-3000 Laser Copier of the company Niceday). The measurement was performed according to DIN EN 1939 with the difference that a rolling weight of 106.7 g was used and application performed by a manual roller, wherein each sample was rolled 4 times in total. This adhesive force is maintained even after several repositioning operations to another part of the sheet. The support film can therefore be repositioned.

For application of this cannula plaster, the adhesive surface of the film structure is released in the first step by removing the siliconized cover paper, as described above. The film structure can then be applied, as described in connection with FIGS. 8a through 8c, wherein the end of the slit (59) is positioned approximately above the point of incision of the cannula. The two wings of the film structure that are covered by the support films (55a, 55b) can be fixed parallel to the cannula. The cannula can be further fixed by these areas. The support film removed during application simultaneously provides the user with a type of notice for documenting the application of the previously applied cannula plaster. Towards this end, merely the cover strip (49) is removed and the support film is glued into the documentation file.

1-14. (canceled)

15. A film structure comprising:
a polymer film having a first side and a second side;
at least one support film disposed on said first side of said polymer film, said support film having a first side facing the polymer film and a second side facing away from the polymer film;
at least one gripping strip formed on said second side of said support film, said support film and said gripping strip cooperating within an application system disposed at said first side of said polymer film for facilitating handling of the film structure; and
at least one adhesive area disposed at said second side of said support film.

16. The film structure of claim 15, wherein said adhesive area is covered by a cover means.

17. The film structure of claim 16, wherein said cover means is strip-shaped.

18. The film structure of claim 15, wherein said polymer film comprises at least one first area without said support film.

19. The film structure of claim 18, wherein said support film comprises at least a first and a second support film portion, disposed in a plane parallel to said polymer film.

20. The film structure of claim 15, wherein said first side of said polymer film has a first and at least one second area without said support film, wherein said second area is separated from said first area.

21. The film structure of claim 20, wherein said first or said second area without said support film forms an edge area of said polymer film.

22. The film structure of claim 20, wherein said first or said second area without said support film is disposed between a first and a second support film portion.

23. The film structure of claim 20, wherein said at least one gripping strip extends over at least sections of said first or said second areas without said support film.

24. The film structure of claim 15, wherein said application system comprises at least two said support films, each having at least one said gripping strip.

25. The film structure of claim 24, wherein one of said two gripping strips extends over at least sections of an other said gripping strip.

26. The film structure of claim 15, wherein said second side of said polymer film, facing away from said support film, is coated with an adhesive, wherein said adhesive is covered by a cover film or a cover paper.

27. The film structure of claim 26, wherein said adhesive extends over an entire surface of said support film.

28. The film structure of claim 15, wherein said polymer film is a polyurethane film, a polyester urethane film, or a polyether urethane film.

29. The film structure of claim 15, wherein the film structure has an incision extending from one edge area to a central area.

30. Use of said support film of the film structure of claim 15 for documentation of application of the film structure.
